(19) United States
(12) Patent Application Publication
(10) Pub. No.: US 2016/0243142 A1
(43) Pub. Date: Aug. 25, 2016

(54) AVICIN D FOR TREATMENT OF MANTLE CELL LYMPHOMA

(71) Applicant: Research Development Foundation, Carson City, NV (US)

(72) Inventors: Jordan U. GUTTERMAN, Houston, TX (US); Valsala HARIDAS, Houston, TX (US)

(73) Assignee: Research Development Foundation, Carson City, NV (US)

(21) Appl. No.: 15/049,843

(22) Filed: Feb. 22, 2016

Related U.S. Application Data
(60) Provisional application No. 62/118,603, filed on Feb. 20, 2015.

(51) Int. Cl. A61K 31/704; A61K 9/00

(52) U.S. Cl. CPC A61K 31/704; A61K 9/0019

(57) ABSTRACT

In some aspects, methods are provided for the treatment of mantle cell lymphoma (MCL) in a patient comprising administering a therapeutically relevant or effective amount of avicin D to the patient.

This application claims the benefit of U.S. Provisional Patent Application No. 62/118,603, filed Feb. 20, 2015, the entirety of which is incorporated herein by reference.

### BACKGROUND OF THE INVENTION

1. Field of the Invention

The present invention relates generally to the field of molecular biology and medicine. More particularly, it concerns treatment of mantle cell lymphoma (MCL) with avicin D.

2. Description of Related Art

Mantle cell lymphoma (MCL), ICD-9-CM diagnosis code 200.4, is a rare and aggressive subtype of B-cell non-Hodgkin lymphoma (NHL). The designation of this subtype was formally presented in the Revised European and American Lymphoma classification (Harris et al., 1994) adopted by the World Health Organization in 2000 (Jaffe et al., 2001). As with most NHL, MCL predominates in males (Harris et al., 1994). When diagnosed, most patients are aged over 60 years and the tumor may have spread to spleen, bone marrow, liver, gastrointestinal tract, and/or the central nervous system. Most MCL patients survive only 3 to 6 years from the date of diagnosis. Although some advances have been made regarding the treatment of MCL, MCL remains a significant and often lethal problem for many patients. Clearly, there exists a need for improved therapies for treating MCL.

### SUMMARY OF THE INVENTION

The present invention is based, in part, on the discovery that avicin D (AVD-001, Avicin) may be used to effectively treat mantle cell lymphoma (MCL) in vivo. In some embodiments, treatment and dosing regimens, as well as related formulations, are provided.

As shown in the below examples, AVD-001 was effective at treating MCL in vivo based on mouse model studies of MCL. The monotherapy efficacy of Avicin for delaying tumor growth in Mino mantle cell lymphoma xenograft bearing NODscid mice was examined. 80 mice were implanted with 1×10⁷ Mino cells, and 50 animals were tumor size rank matched into five treatments groups of 9-10 animals each with ˜150 mm³ mean tumor volumes. The three test cohorts were dosed subcutaneously (s.c.) on days 1-33 with 0.5 mg/kg Avicin (Group 3), on days 1-2 and 5-33 with 1 mg/kg Avicin (Group 4), or on days 1-2 and 5-33 with escalating 1.0-2.0 mg/kg Avicin (Group 5). The two control cohorts were dosed i.v. on days 1 and 8 and s.c. on days 1-33 with saline vehicle (Group 1) or i.v. on days 1 and 8 with 3.3 mg/kg Adriamycin positive control (Group 2). Tumor volumes and body weights were measured three times weekly throughout the duration of the study. The study tumor endpoint was set at 2000 mm³, and the tumor growth delay (TGD) method was used to determine treatment efficacy. Log-rank two-tailed statistical analysis with a 95% confidence was used to determine the significance of tumor response comparisons between treatment groups and Log₁₀ cell kill analysis was used to indicate Avicin antitumor activity. The Avicin dosing protocols were generally well tolerated as animals gained weight progressively throughout the study. Avicin was administered to Group 3 animals without interruption, while Avicin administration in Groups 4 and 5 was temporarily suspended (day 3 and 4 only) because of transient 10% body weight loss. Two animals in the escalating Avicin dose group (Group 5) were removed from the study on day 34 and day 38 respectively due to drug toxicity, but the remaining Group 5 animals reached the 2000 mm³ tumor endpoint. All animals in Groups 1, 3, and 4 reached the 2000 mm³ tumor endpoint. The escalating 1.0-2.0 mg/kg Avicin dose (Group 5) was most efficacious in inhibiting Mino tumor growth. The corresponding 76.2% TGD was nearly double the 44.1% TGD for the 1.0 mg/kg dose (Group 4) and more than double the 34.9% TGD for the 0.5 mg/kg dose (Group 3). Log-rank analysis showed significant Avicin therapeutic efficacy compared to vehicle control, with Group 5 having the highest significance (p=0.0004) and Groups 3 (p=0.03) and 4 (p=0.02) having lower significance. Log-rank analysis also showed a significant dose response (p=0.004) between the escalating 1.0-2.0 mg/kg Avicin dose (Group 5) and the 0.5 mg/kg dose (Group 3). Log-rank analyses comparing Avicin treatment Group 3 vs Group 4 and Group 4 vs. Group 5 did not show significant dose responses (p=0.3394 and p=0.3097, respectively). Thus, monotherapy with Avicin showed significant tumor growth delay in Mino mantle cell lymphoma xenograft bearing NODscid mice. There was high statistical significance in comparisons of escalating 1.0-2.0 mg/kg Avicin treatment responses with vehicle controls. In addition, a Logio cell kill value of 0.89 in the 1.0-2.0 mg/kg cohort indicated antitumor efficacy.

An aspect of the present invention relates to a method of treating mantle cell lymphoma (MCL) in a subject in need of said treatment, comprising administering a pharmacologically effective amount of avicin D to the subject, wherein the subject has mantle cell lymphoma (MCL). The subject may be a mammal such as, e.g., a human. In some embodiments, said amount is from about 0.0125 mg/kg to about 0.1 mg/kg. In some embodiments, said amount is from about 0.0125 mg/kg to about 2 mg/kg, from about 0.0125 mg/kg to about 1 mg/kg, from about 0.025 mg/kg to about 0.75 mg/kg, from about 0.05 mg/kg to about 0.5 mg/kg, or from about 0.0125 mg/kg to about 0.1 mg/kg. In some embodiments, about 0.0125-0.050, about 0.0125-0.050, or about 0.0125-0.025 mg/kg/day, or any range derivable therein, may be administered to a human patient to treat MCL. In some embodiments, the avicin D is comprised in a pharmaceutically acceptable excipient or diluent. The pharmaceutically acceptable excipient or diluent may be formulated for injection or oral administration. In some embodiments, said injection is intravenous (i.v.), subcutaneous (s.q.), intracutaneous (i.e.), intramuscular (i.m.), or intraperitoneal (i.p.). In some embodiments, said avicin D is administered prior to, after, or in combination with another anti-cancer agent, an anti-cancer therapy, or a surgery. The anti-cancer agent may be a chemotherapeutic. In some embodiments, the anti-cancer agent is cyclophosphamide, hydrozydaunorubicin, vincristine, prednisone, rituximab, cytarabine, dexamethasone, cytarabine, cisplatin, or bendamustine. In some embodiments, avicin D is be administered prior to, after, or in combination with an anti-cancer therapy, wherein the anti-cancer therapy is CHOP, R-CHOP, R-bendamustine, DHAP, R-DHAP, or R-Hyper-CVAD/MA. Said MCL in said subject may be resistant to a chemotherapy (e.g., cyclophosphamide, hydrozydaunorubicin, vincristine, prednisone, rituximab, cytarabine, dexamethasone, cytarabine, cisplatin, or bendamustine). In some embodiments, said MCL in said subject is resistant to an anti-cancer therapy (e.g., CHOP, R-CHOP, R-bendamustine, DHAP, R-DHAP, or R-Hyper-CVAD/MA). In some embodiments, the method is further defined as a method of overcoming chemotherapeutic resistance of said MCL to an anti-cancer treatment. The anti-cancer treatment may be a stem cell therapy such as, e.g., an autologous stem cell therapy. In some embodiments, growth rates of tumors of said MCL in the subject decrease. In some embodiments, the total tumor volume resulting from said MCL in the subject decrease. In some embodiments, said MCL is a classic MCL or a nodular MCL. In some embodiments, said MCL is a diffuse MCL or a blastoid MCL. The avicin D may be administered to the subject in an amount of from about 0.0125 to about 0.1 mg/kg per day. In some embodiments, the avicin D is administered intravenously or subcutaneously.

Another aspect of the present invention relates to use of avicin D for the treatment of a subject having mantle cell carcinoma (MCL). The subject may be a mammalian subject such as, e.g., a human.

Yet another aspect of the present invention relates to a pharmaceutical preparation comprising avicin D for the treatment of a subject having mantle cell carcinoma (MCL). The subject may be a mammalian subject such as, e.g., a human.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: AVD-001 Efficacy Evaluation shown as mean tumor volume responses.

FIG. 2: AVD-001 Efficacy Evaluation shown as median tumor volume responses.

FIG. 3: Mean percent body weight changes of mice are shown.

FIG. 4. Time to Tumor Endpoint Values.

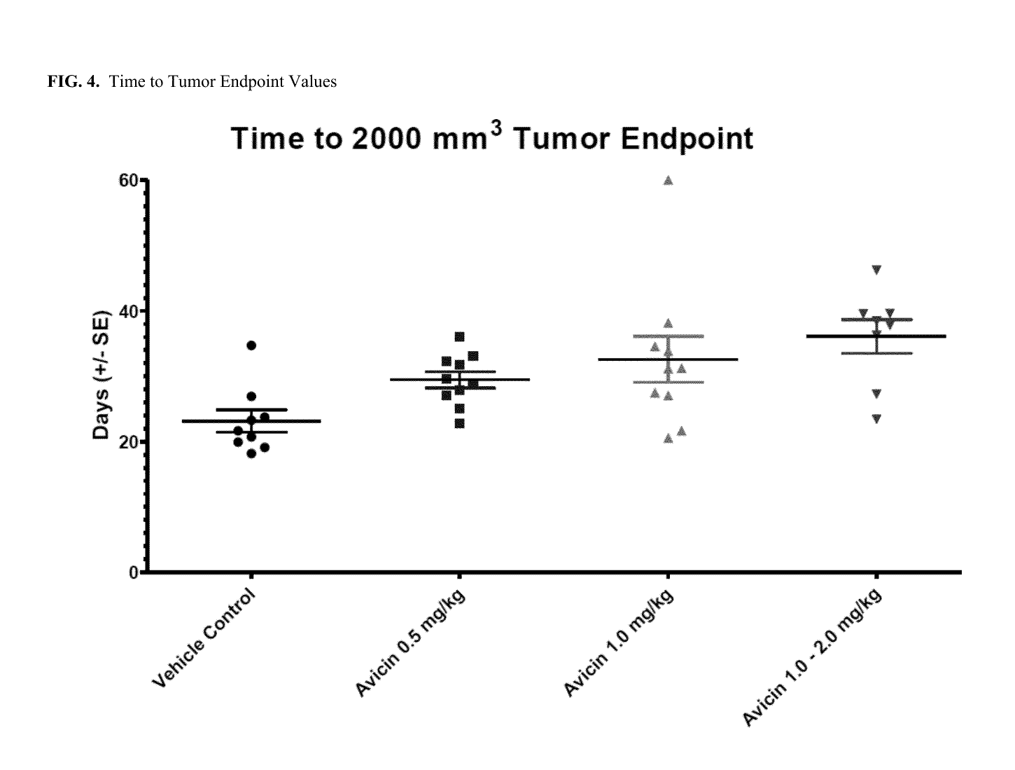

FIG. 5: Kaplan-Meier Plots for Avicin Treatment Responses.

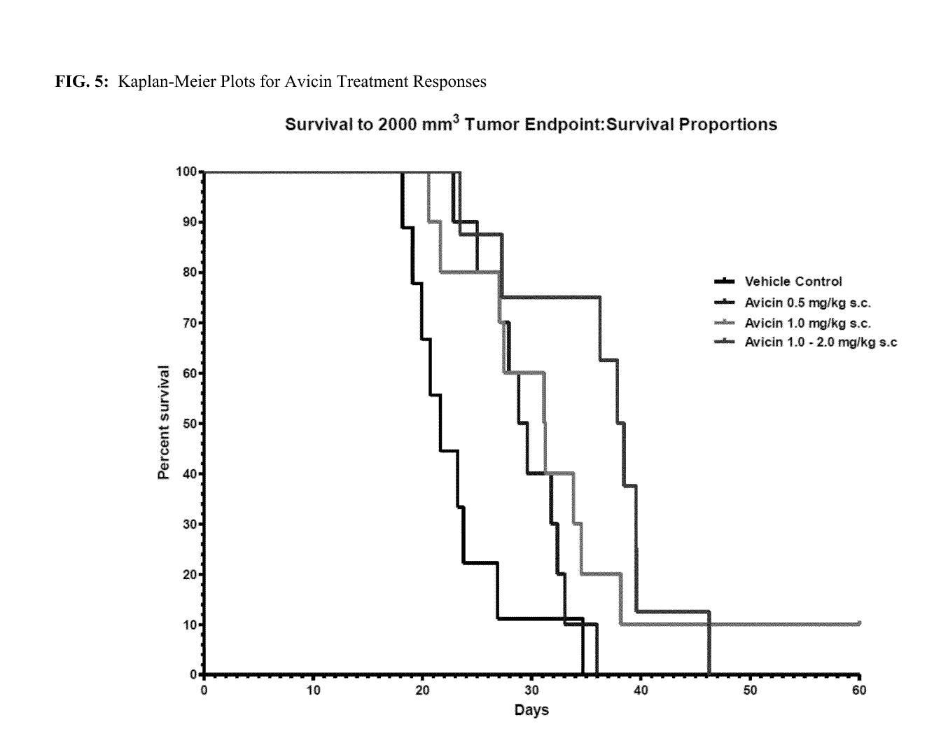

### DESCRIPTION OF ILLUSTRATIVE EMBODIMENTS

I. Mantle Cell Lymphoma

Mantle cell lymphoma (MCL), ICD-9-CM diagnosis code 200.4, is a rare and aggressive subtype of B cell non Hodgkin lymphoma (NHL). MCL typically arises in the mantle zone of the lymphoid follicle. MCL comprises approximately 5% to 10% of NHL cases (Leux et al., 2014; Smedby and Hjalgrim, 2011). The designation of this subtype was formally presented in the Revised European and American Lymphoma classification (Harris et al., 1994) adopted by the World Health Organization in 2000 (Jaffe et al., 2001). It is anticipated that, in various embodiments, avicin D may be administered to a patient with MCL at any time between the patient's initial diagnosis and remaining lifespan.

A hallmark of MCL is a translocation between chromosomes 11 and 14 t(11:14)(q13;q32). With this translocation, the B-cell leukemia/lymphoma-1 (bcl-1) gene, also known as CCND1, is regulated by the immunoglobulin heavy chain enhancer which leads to overexpression of the CCND1 gene encoding Cyclin D1. Cyclin D1 is a protein involved in cell division and in cell cycle progression from G1 to S phase. Cyclin D1 is not overexpressed in most other NHLs and little or no expression is seen in normal lymphoid cells (de Boer et al., 1995).

Defects in cell cycle regulation, apoptotic pathways, DNA repair mechanisms, and cell signaling are also hallmarks of MCL (Jares et al., 2012). The INK4a/CDK4/RB1 and ARF/MDM2/p53 pathways are frequently altered in MCL. The CDKN2A locus that encodes both INK4a and ARF has been found to be deleted in some MCLs. Point mutations in the RB1 and p53 checkpoint genes are also often found in MCL (Pinyol et al., 2007; Hernandez et al. 1996). MDM2, another pathway member, often undergoes gene amplification in MCL (Hernandez et al., 2005). Cyclin D1 expression in MCL cells also has been shown to sequester the proapoptotic protein BAX, potentially promoting cell survival (Beltran et al., 2011). These MCL characteristics are reviewed in Jares et al. (Jares et al., 2012).

Another important signalling pathway often found to be hyperactivated in MCL is the mammalian target of rapamycin (mTOR) pathway, which controls both cell death and growth functions. The mTOR activator AKT, the mTOR protein itself, and mTOR downstream targets are often upregulated, while the activation phosphatase and tensin homolog (PTEN), a negative regulator of mTOR, is often downregulated or inactivated in MCL.

Pro-oncogenic transcription factor pathways can also be disrupted in MCL. Phosphorylated signal transducer and activator of transcription 3 (STAT3) is sometimes seen in primary MCL (Baran-Marszak et al., 2010; Lai et al., 2003). The Jak/STAT3 pathway functions as a cell survival and proliferation signal and phosphorylated STAT3 is the transcriptionally active form of the protein. Similarly, the nuclear factor-κB (NF-κB) pathway has been found to be constitutively active in some MCLs (Roue et al., 2007; Pham et al., 2003). The activation of the NF κB pathway can lead to the expression of a number of antiapoptotic proteins such as x-linked inhibitor of apoptosis protein (XIAP).

All non-Hodgkin varieties of lymphoid neoplasms are histologically characterized by the distinctive absence of Reed-Sternberg giant cells. MCL is a subtype of B-cell-derived NHLs that generally histologically presents with a homogeneous population of CD5-positive, antigen-naive, pre-germinal centre B-cells within the mantle zone that surrounds normal germinal centre follicles. MCLs classically over express cyclin-D1, which is a phenomenon attributed to a translocation between chromosomes 11 and 14.

Diagnosis of a patient with MCL often occurs at an advanced stage of MCL. Traditional evaluation techniques used to diagnose MCL may include lymph node aspiration or biopsy, bone marrow aspiration or biopsy, immunophenotyping for differential diagnosis, and/or full body computed tomography scan for initial staging. Hematological studies (e.g., complete blood count, serum chemistry, liver function tests, (β2-microglobulin, and/or immunophenotyping) may also be used for refining the prognosis or treatment regime. Symptoms of MCL are similar to many other hematological malignancies and may include: B symptoms, which include fever, night sweats, and weight loss, in 40% of patients; generalized lymphadenopathy; abdominal distention from hepatosplenomegaly; and/or fatigue from anemia or bulky disease.

Four morphological subtypes of MCL are recognized. These subtypes include classic MCL, nodular MCL, diffuse MCL, and blastoid MCL. Classic or mantle zone MCL presents in the typical mantle zone invasion pattern. Diffuse MCL presents a less localized pattern and nodular MCL presents a histology intermediate between classic and diffuse MCL. Blastoid MCL is the most aggressive MCL and is characterized by larger cells with more diffuse nuclear staining.

II. Avicin D

Avicin D (also referred to herein as AVD-001) is classified as a member of the avicin family of triterpenoid saponin from the saponin group of compounds. AVD-001 is extracted from the seed pods of the desert legume Acacia victoriae. The molecular formula of AVD-001 is C₉₈H₁₅₅NO₄₆ and the monoisotopic mass is 2081.98 Da. Its structure is characterized by acacic acid-bearing oligosaccharides at C-3 and C-28 and a side chain (linked to C-21) comprising of two monoterpene carboxylic acids and a quinovose moiety. Avicin D has the chemical name: [(2S,3R,4S,5S,6R)-3-[(2S,3R,4S,5S,6S)-5-[(2S,3R,4R,5S)-3,4-dihydroxy-5-(hydroxymethyl)oxolan-2-yl]oxy-3-hydroxy-6-methyl-4-[(2S,3R,4S,5S,6R)-3,4,5-trihydroxy-6-(hydroxymethyl)oxan-2-yl]oxyoxan-2-yl]oxy-4,5-dihydroxy-6-(hydroxymethyl)oxan-2-yl] (3S,4aR,5R,6aR,6aS,6bR,8aR,10S,12aR,14bS)-10-[(2R,3R,4R,5S,6R)-3-acetamido-6-[[(2R,3R,4S,5R,6R)-4,5 -dihydroxy-6-methyl-3-[(2S,3R,4S, 5R)-3,4,5-trihydroxyoxan-2-yl]oxyoxan-2-yl]oxymethyl]-4,5 -dihydroxyoxan-2-yl]oxy-3-[(2E,6R)-6-[(2S,3R,4R,5S,6R)-3,4-dihydroxy-5-[(2E,6R)-6-hydroxy-2-(hydroxymethyl)-6-methylocta-2,7-dienoyl]oxy-6-methyloxan-2-yl]oxy-2-(hydroxymethyl)-6-methylocta-2,7-dienoyl]oxy-5-hydroxy-2,2,6a,6b,9,9,12a-heptamethyl-1,3,4,5,6,6a,7,8,8a,10,11,12,13,14b-tetradecahydropicene-4a-carboxylate. Avicin D has the structure:

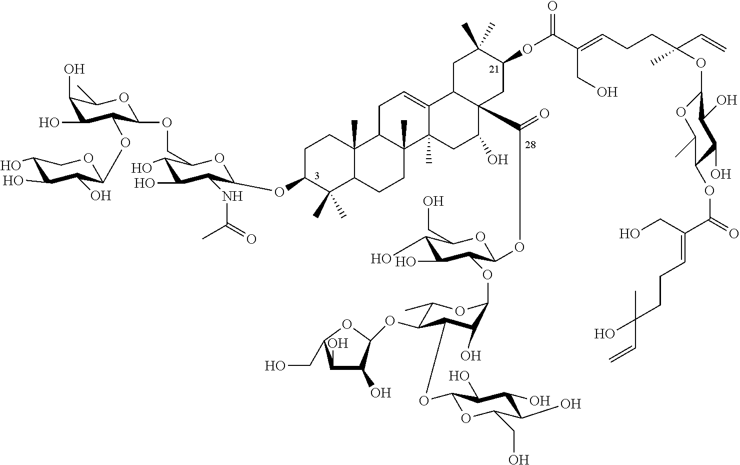

Additional formulations of avicin D that may be used with the present invention include those described in, e.g., WO2013126730 (PCT/US2013/027362).

Without wishing to be bound by any theory, evidence suggests that AVD-001 has been shaped by evolution, possibly as a defense by plants against external predators (Blackstone et al., 2005). The molecule can alter both information flow (signal transduction) and energy transfer (metabolism) required to sustain tumor growth. Since most tumors show a myriad of genetic and epigenetic changes, AVD-001's bimodal effect on cancer cells, in particular the suppression of excess tumor cell energetics (common to all cancers), may make this compound particularly useful for the treatment of cancer.

AVD-001 displays several anti-cancer cellular effects. AVD-001 suppresses proliferation and induces apoptosis of a wide variety of solid tumor and hematological tumor types in cell culture (Jayatilake et al., 2003; Haridas et al., 2001; Mujoo et al., 2001; Zhang et al., 2008; Mitsiades et al., 2004; Haridas et al., 2009). Dose concentrations producing 50% inhibition range from low to mid nanomolar (200 to 500 nM) for hematologic tumors and from mid to high nanomolar (500 to 900 nM) for most solid tumor types. Without wishing to be bound by any theory, evidence supports the idea that the process of cell death is facilitated by AVD-001's effects on both mitochondrial (energy) and nuclear/cytoplasmic (information) compartments. In mitochondria, avicins induce closure of the voltage-dependent anion channel, permeabilize the outer mitochondrial membrane, and allow release of cytochrome C to initiate the intrinsic caspase pathway (Jayatilake et al., 2003; Haridas et al., 2001; Mujoo et al., 2001; Zhang et al., 2008; Mitsiades et al., 2004; Haridas et al., 2009; Gaikwad et al., 2005; Haridas et al., 2007; Lemeshko et al., 2006). During this process, two anti-apoptotic proteins, heat shock protein-70 and XIAP, are downregulated by ubiquitination (Gaikwad et al., 2005). XIAP is one of the downstream targets of the NF-KB pathway that is implicated in antiapoptotic responses in MCL.

Furthermore, AVD-001 can suppress functional expression of several pro-survival oncogenic proteins involved in transcription and signal transcription, several of which are implicated in the pathogenesis of MCL. These pathways include NF-κB (Haridas et al., 2001), phosphatidylinositol-3-kinase and AKT (Mujoo et al., 2001), STAT3, cMyc, and cyclin D1 (the gene whose aberrant expression in B cells is the hallmark of MCL (Zhang et al., 2008; Haridas et al., 2009). AVD-001 can cause posttranslational changes, including suppression of phosphorylation (Haridas et al., 2009), thiol modification (Haridas et al., 2005; Haridas et al., 2004), ubiquitination (Gaikwad et al., 2005; Gutterman et al., 2005), and acetylation-deacetylation. For example, AVD-001 can decrease serine/threonine and tyrosine phosphorylation of AKT and STAT3 (Mujoo et al., 2001; Haridas et al., 2009). This decrease in phosphorylation may be due to decreased levels of available adenosine triphosphate (ATP) and activation of PP1, a phosphatase. Thiol modification can occur in the suppression of NF-κB (Haridas et al., 2001). Thus, excess oncogene signalling can be dampened by suppressing energy sources necessary for function, and alterations in tumor metabolism can affect gene function.

AVD-001's activity in mitochondria, the main source of bioenergy via ATP for the cell, can directly affect the process of tumor cell apoptosis. AVD-001 can suppress oxygen consumption, reduce ATP levels, and accelerate formation of mitochondrial reactive radical species, helping activate cell death pathways (Haridas et al., 2007; Lemeshko et al., 2006). Mitochondrial effects of AVD-001 may be augmented by Warburg-like suppression of glycolysis due to reduction in Glut-1 and inhibition of glucose uptake. Lipogenesis can also be impaired by downregulation of fatty acid synthase in prostate cancer cells in vitro and in vivo. Thus, avicins may induce a broad effect on tumor biology by suppressing glycolysis, oxidative phosphorylation, and lipogenesis. Inappropriate activation of the mTOR pathway may be implicated in the pathogenesis of many MCLs. AVD-001 may activate adenosine monophosphate-activated protein kinase which, in turn, may downregulate mTOR and S6 kinase activity. Through this mechanism, aberrant tumor metabolism may be further altered to promote impairment of amino acid metabolism and induction of autophagy (Xu et al., 2007).

AVD-001 suppresses proliferation and induces apoptosis in various solid and haematological tumor types in cell culture. Dose concentrations producing 50% inhibition of proliferation range from low to mid nanomolar (e.g., about 200 to 500 nM) for haematological tumors to mid to high nanomolar (e.g., 500 to 900 nM) for most solid tumor types. As noted above, the process of cell death may be facilitated by AVD-001′s effect on nuclear-cytoplasmic signalling and on energy production in mitochondria.

In contrast to its activity against tumor cells, AVD-001 appears to protect nonmalignant, quiescent cells against cellular stress in vitro through induction of the thiol-regulated transcription factor NF-E2-related factor 2. A variety of phase 2 enzymes and proteins responsible for cellular defense may be induced, including glutathione peroxidase, ferritin, bilirubin, and heme-oxygenase (Haridas et al., 2004).

In some embodiments, AVD-001 medicinal product is provided as a sterile solution, e.g., prepared at dose strengths of 0.5 mg/mL and 2.0 mg/mL. The formulation may contain AVD-001 diluted in a sodium chloride saline solution for injection buffered with sodium acetate to a pH of 4.5±0.2 aseptically filled at a volume of 2.5 mL into a 5 mL Type I glass vial closed with a rubber stopper and aluminium flip-off seal.

In some embodiments, greater than 0.125 mg/kg, greater than 0.25 mg/kg, or greater than 0.5 mg/kg may be administered to the subject. In some embodiments, about 0.0125, 0.02, 0.025, 0.03, 0.04, 0.05, 0.06, 0.07, 0.075, 0.08, 0.09, 0.1, 0.125, 0.25, 0.5, 1.0, 1.25, 1.5, 1.75, 2.0, 2.25, 2.5, 2.75, 3.0, 3.5, 4.0, 4.5, 5.0, 6.0, 7.0, 8.0, 9.0, to 10.0 mg/kg, or any range derivable therein, of avicin D may be administered to a mammalian subject (e.g., a mouse, rat, primate, monkey, ape, or human). In some embodiments, from about 0.0125 mg/kg/day to about 0.10 mg/kg/day of avicin D may be administered to a human patient to treat MCL. In some embodiments, about 0.0125-0.050, about 0.0125-0.050, or about 0.0125-0.025 mg/kg/day, or any range derivable therein, may be administered to a human patient to treat MCL. In some embodiments, the AVD-001 is administered subcutaneously or intravenously. Optionally, lidocaine may be administered at the injection site to treat any inflammation or pain observed at the injection site. In some embodiments, avicin D is delivered by subcutaneous administration. In some embodiments, a buffer may be included in the pharmaceutical composition to reduce the acidity or increase the alkalinity of the pharmaceutical composition comprising avicin D.

IV. Combination Therapies

In some embodiments, AVD-001 may be administered in combination with one or more additional cancer therapies to treat MCL. Additional cancer therapie(s) may vary treatment for MCL, depending on stage of disease and status of the patient. The current standard for staging of MCL is also used for NHL, as shown in Table 1. MCL is often diagnosed at Stage 3 or Stage 4 and is treated as an aggressive malignancy.

TABLE 1
Staging of Mantle Cell Lymphoma

| Stage | Criteria |
|---|---|
| 1 | One group of lymph nodes affected |
| 2 | Two or more groups of lymph nodes affected and are on the same |
| | side of the diaphragm (above or below) |
| 3 | In lymph nodes above and below the diaphragm |
| 4 | Spread to other organs besides lymph nodes |

The Ki-67 index has been proposed as a marker for rapidity of spread of disease, but inter-institutional and inter-study reliability has not been established. By contrast, the MCL International Prognostic Index (MIPI) provides the most reliable and validated outcome measure (Hoster et al., 2008). In some embodiments, the MCL MIPI may be used to further diagnose MCL in a patient. The MIPI incorporates the factors of age, Eastern Cooperative Oncology Group performance status, serum lactate dehydrogenase, and white blood cell count to provide information that will aid treatment decisions for patients with advanced stage MCL.

Subjects assessed by MIPI may be divided into three groups with different treatment strategies: elderly, fit patients; elderly, frail patients; and younger patients (aged <65 years). In some embodiments, AVD-001 may be used in combination with another therapeutic, e.g., as described below for MCL in a particular subset of patients as identified using MIPI.

In newly diagnosed patients, an induction chemotherapy regimen may be used that includes the cyclophosphamide, hydroxydaunorubicin, vincristine, and prednisone (CHOP) combination, with or without rituximab. Other inductions may use cytarabine alone, or dexamethasone, cytarabine, and cisplatin combinations. Administration of bendamustine alone or in combination with other agents may be used in various embodiments. Table 2 outlines some of the common induction regimens for MCL that may further include AVD-001. It is anticipated that inclusion of AVD-001 in combination with one or more additional therapeutics, e.g., as described below, may allow for either (1) exclusion of one or more of the agents typically used to treat MCL, and/or (2) lower dosages to be used to treat the MCL. In some embodiments, AVD-001 may be administered as a monotherapy to treat MCL in a patient.

TABLE 2
Common Induction Regimens for Mantle Cell Lymphoma

| Abbreviation | Medications |
|---|---|
| CHOP | cyclophosphamide, doxorubicin, vincristine, and |
| | prednisone |
| R-CHOP | rituximab with cyclophosphamide, doxorubicin, |
| | vincristine, and prednisone |
| R bendamustine | rituximab with bendamustine |
| DHAP | dexamethasone, high-dose cytarabine, and cisplatin |
| R-DHAP | rituximab with dexamethasone, high-dose cytarabine, |
| | and cisplatin |
| R-Hyper- | rituximab with fractionated cyclophosphamide, |
| CVAD/MA | doxorubicin, vincristine, dexamethasone; |
| | alternated with high-dose methotrexate |
| | and cytarabine |

After an induction therapy comprising administration of AVD-001 to a patient with MCL, the patient may be maintained with interferon, or if appropriate, with an autologous stem cell transplantation. In some embodiments, a patient may be maintained on AVD-001 after an initial therapy involving administration of AVD-001 in combination with one or more agents, e.g., as described above. In some embodiments, an initial induction therapy may be performed, e.g., as described above, but without inclusion of AVD-001 with the other therapeutics, and then the patient may then be maintained on AVD-001 after the initial induction therapy.

Some therapies have been developed that can be used specifically in relapsed patients. For example, temsirolimus, which blocks mTOR, has been approved by European Medicines Agency (EMA) to treat relapsed MCL patients. In some embodiments, AVD-001 may be used in combination with temsirolimus to treat MCL in a relapsed patient. In clinical trials, patients receiving the approved dose of Torisel lived for an average of 4.8 months without their disease getting worse; in comparison, the average was 1.9 months in patients receiving the alternative treatment. In the United States (U.S.), Velcade™ (bortezomib), Revlimid™ (lenalidomide), and Imbruvica™ (ibrutinib) have been approved for treatment of MCL in a relapsed patient; in some embodiments, AVD-001 may be used in combination with one or more of these agents to treat MCL in a relapsed patient. Bortezomib is a reversible inhibitor of the chymotrypsin-like activity of the 26S proteasome in mammalian cells and has been approved by the EMA for treatment of multiple myeloma. Lenalidomide is an analogue of thalidomide with immunomodulatory, anti-angiogenic, and anti-neoplastic properties and has been approved by the EMA for the treatment of multiple myeloma. Ibrutinib is a small molecule inhibitor of Bruton's tyrosine kinase and data have been submitted by the sponsor for the treatment chronic lymphocytic leukemia, small lymphocytic leukemia, and MCL. Based on the current available data, all three drugs appear to show approximately a similar capacity to improve the overall response rate and duration of response in relapsed cancers; however, limited information is available on the effect of these therapies on overall survival in patients with MCL.

In some embodiments, AVD-001 may be administered before or after an autologous stem cell replacement, to treat MCL in a patient. Stem cell replacement has been used effectively to treat MCL in patients, but this therapy currently remains as an option for only a very small percentage of the MCL population. First, the patient must generally be in much better health than the typical MCL patient to have a chance at success with this therapy. Second, this level of treatment is only available at a limited number of facilities. Consequently, the advanced age and fragility of patients with MCL, compounded by lack of access to facilities with appropriate resources, means that stem cell replacement therapy typically does not represent a satisfactory method of treatment for the broader MCL population. Nonetheless, in patients who may qualify for treatment with a stem cell replacement therapy, AVD-001 may advantageously be administered.

V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Mice and Housing

80 NOD .CB17 -Prkdcscid/J (NOD scid) (JAX, West Sacramento, Calif.) 4-6 week old female mice were transferred to the in vivo research laboratory in West Sacramento, CA. The mice were ear notched for identification and housed in individually and positively ventilated polycarbonate cages with HEPA filtered air at a density of five mice per cage. Bed-o'cobs® bedding was used and cages were changed every two weeks. The animal room was illuminated entirely with artificial fluorescent lighting, with a controlled 12 h light/dark cycle (7 am to 7 pm light). The normal temperature and relative humidity ranges in the animal rooms were 22±4° C. and 50±15%, respectively. The animal rooms were set to have 15 air exchanges per hour. Filtered tap water, acidified to a pH of 2.8 to 3.1, and LabDiet 5LL4 were provided ad libitum.

The following solutions were prepared:

| | |
|---|---|
| Id. (Name) | AvicinD |
| Storage | 4° C. |
| Source | Qwell |
| Formulation | 1. 2.0 mg Avicin added to 1.0 ml 100% ethanol and heated and |
| | sonicated until dissolved. |
| | 2. 9 ml water added to Step 1 for 0.2 mg/ml stock solution in 10% |
| | EtOH. |
| | 3. Group 3: 1 ml stock (Step 2) added to 3 ml water for a 0.05 mg/ml |
| | solution. |
| | 4. Group 4: 1 ml stock (Step 2) added to 1 ml water for a 0.1 mg/ml |
| | solution. |
| | 5. Group 5: 0.1 mg/ml solution prepared as in Step 4 (above) for 1 mg/ml |
| | dosing. 1.5 ml stock (Step 2) added to 0.5 ml water for a |
| | 0.15 mg/ml solution for 1.5 mg/kg dosing, and 0.2 mg/ml stock |
| | (Step 2) used for 2 mg/kg dosing. |
| Concentration | 0.5 mg/ml, 0.1 mg/ml, 0.15 mg/ml, 0.2 mg/ml |
| Id. (Name) | Adriamycin |
| Storage | 4° C. |
| Source | Bedford Labs |
| Formulation | 10 mg/6 ml |
| Concentration | 1.67 mg/ml |

Tumor Implantation

Mino Mantle Cell Lymphoma cells (ATCC catalogue #CRL-3000™, Manassas, Va.) were maintained in RPMI 1640 with 2 mM 1-glutamine, 25 mM HEPES, 4.5 g/L D-glucose (Invitrogen, San Diego, Calif.) supplemented with 10% ES cell certified fetal bovine serum (Hyclone, Logan, Utah). The cells were suspended at 1×10⁸ cells/ml in serum free media, and 80 mice were subcutaneously injected in the rear right flanks with 10⁷ cells in 100 μL. Tumor volumes were monitored with ULTRA-Cal IV digital calipers (Fowler, Newton, Mass.), and 50 animals were tumor size rank matched into five cohorts of 10 animals with mean tumor volumes of approximately 150 mm³.

Tumor Implantation

Mino Mantle Cell Lymphoma cells (ATCC catalogue #CRL-3000™, Manassas, Va.) were maintained in RPMI 1640 with 2 mM 1-glutamine, 25 mM HEPES, 4.5 g/L D-glucose (Invitrogen, San Diego, Calif.) supplemented with 10% ES cell certified fetal bovine serum (Hyclone, Logan, Utah). The cells were suspended at 1×10⁸ cells/ml in serum free media, and 80 mice were subcutaneously injected in the rear right flanks with 10⁷ cells in 100 μL. Tumor volumes were monitored with ULTRA-Cal IV digital calipers (Fowler, Newton, Mass.), and 50 animals were tumor size rank matched into five cohorts of 10 animals with mean tumor volumes of approximately 150 mm³.

Treatment Protocol

Animals were treated according to Table 3 below. Animals were dosed in the opposite flank opposing the tumors at 10 mL/kg. Daily clinical observations were made and body weights and tumor measurements were performed thrice weekly. Animals were maintained on study until their tumor burden reached the study endpoint of 2000 mm³. Animals were euthanized at the study endpoint with terminal cardiocentesis performed for serum collection and plasma preparation in K₂EDTA. Tumors were excised and fixed in 10% formalin.

TABLE 3
Treatment Group Information

| Group | | | Dose | | Days | Dose |
|---|---|---|---|---|---|---|
| (Cohort) | N | Treatment | (mg/kg) | Schedule | Dosed | Route |
| 1 | 9 | Vehicle | 5.0 | Weekly | 1 and 8 | i.v. |
| | | control | | Daily | 1-33 | s.c. |
| 2 | 10 | Adriamycin | 3.3 | Weekly | 1 and 8 | i.v. |
| 3 | 10 | AVD-001 | 0.5 | Daily | 1-33 | s.c. |
| 4 | 10 | AVD-001 | 1.0 | Daily | 1-2 | s.c. |
| | | | | | 5-33 | |
| 5 | 10 | AVD-001 | 1.0 | Daily | 1-2, 5-10 | s.c. |
| | | | 1.5 | | 11-15 | |
| | | | 2.0 | | 16-33 | |

i.v. = intravenously,
s.c. = subcutaneously

Specimen and Data Collection

Animals were euthanized when the tumors had reached the 2000 mm³ study endpoint, and the tumors were harvested, weighed, and placed in 10% formalin. Table 4 summarizes the clinical observation schedule and the body weight and tumor measurement schedule performed.

TABLE 4
Clinical Observations Schedule

| | Observation | |
|---|---|---|
| Result Type | Schedule | Storage Site |
| Morbidity and | Daily cage side | Raw data archived in study folder^{a} |
| Mortality | observation with | |
| | clinical observation | |
| | 3X weekly | |
| Body Weights | 3X weekly upon | Raw data archived in study folder^{a}, |
| | study initiation | electronic version in section 7.2 |
| Tumor | 3X times weekly | Raw data archived in study folder^{a}, |
| Measurements | during study. | electronic version in section 7.2 |

Statistical Tumor Analysis

Tumor volumes were calculated from digital caliper raw data by using the formula:

$$Volume ( {mm}^{3} ) = \frac{( l \times {w}^{2} )}{2} .$$

The value w (width) was the smaller of two perpendicular tumor axes and the value l (length) was the larger of two perpendicular axes.

The tumor growth delay (TGD) method was used to analyze drug treatment effects on time (days) to tumor endpoint (TTE) for Mino tumor response. The tumor endpoint volume for TTE analysis was set at 2000 mm³ and TTE was defined in days by the formula:

$$TTE ( days ) = \frac{{log}_{10} ( 2000 ) - b}{m} .$$

The value for b was the (y) intercept and m was the slope of the line calculated from a linear regression of log-transformed tumor growth data for each tumor calculated from a minimum of three time points preceding 2000 mm³ and a minimum of one that surpassed the time point for 2000 mm³. Treatment initiated when tumors reached approximately 150 mm³, and animals were drugged according to Table 3. Tumors were monitored until animals were euthanized due to tumors reaching a 2000 mm³ endpoint, and the study was concluded on day 59 post drug treatment initiation.

Treatment efficacy was determined by comparing TTE values calculated for each treatment group and by calculating percent tumor growth delay for each treatment group:

$$\% TGD = \frac{( T - C )}{C} \times 100.$$

T represented the median TTE in days for a drug treatment group and C represented the median TTE in days for the control group. Statistical significance for median TTE values for treatment group comparisons was determined by the Log-rank test with GraphPad Prism 5.0 software (GraphPad, La Jolla, Calif.). A 95% confidence value was used for two-tailed statistical analyses. Log-rank survival curves were plotted based on survival to 2000 mm3 to visualize the statistical significance of the median TTE values between treatment groups.

Median tumor volume growth curves and mean tumor volume growth curves with standard error were calculated for each treatment group. The values excluded euthanized animals one time point after the day of euthanasia and the curves were truncated when >50% of the starting cohort animals reached the tumor endpoint. Kaplan-Meier survival plots showing the percentage of animals surviving at particular time points were generated from the TTE data with GraphPad Prism 5.0.

Complete regression (CR) was recorded as tumor shrinkage below measurable size for three consecutive time points. Partial regression (PR) was recorded as a <50% reduction from initial tumor size for three consecutive time points. CR responses through the study endpoint were recorded as tumor free survivors (TFS).

The Logic) cell kill for treatment Groups three, four, and five was determined by the formula:

$${Log}_{10} cell kill = \frac{( T - C )}{3.32 \times {T}_{d}} \times 100.$$

The value T is the median TTE (days) for tumors to reach a tumor burden of 750 mm³ for the treatment groups. The value C is the median TTE (days) for tumors in the control group to reach tumor burden of 750 mm³. Tumors that failed to reach the 750 mm³ mid log phase value were excluded from analysis. Td represents the time in days for control animal tumors to double in volume during exponential growth phase. This was calculated from a non-linear regression analysis with GraphPad 5.0. A compound was considered to have activity in a given model when the value obtained from the Logio cell kill was greater than or equal to 0.7.

Toxicity

Non-treatment related deaths (NTR), non-treatment metastatic related deaths (NTRm), and treatment related deaths (TR) were monitored. Animal body weight was monitored three times weekly throughout the study and mean % body weight loss was calculated and plotted with GraphPad Prism 5.0. Animals displaying >20% body weight loss were to be euthanized and recorded as TRs.

### EXAMPLE 2

### Avicin D (AVD-001) for the Treatment of MCL

The monotherapy efficacy of AVD-001 for delaying tumor growth in Mino MCL xenograft bearing NODscid mice was examined. A summary of the results are shown below.

A total of 80 mice were implanted with 1×10⁷ Mino cells. From that group 50 animals were tumor size rank matched into five treatments groups of 9 to 10 animals, with each carrying approximately 150 mm³ mean tumor volumes. Tumors were allowed to establish up to the 150 mm³ size prior to initiation of treatment. As noted above, one treatment cohort received only vehicle (Group 1), one received active comparator of Adriamycin (doxorubicin) (Group 2), and three received differing doses of AVD-001 (Group 3, Group 4, and Group 5).

Tumor volumes and body weights were measured three times weekly throughout the study. The tumor endpoint for the study was set at 2000 mm³, and the tumor growth delay (TGD) method was used to compare treatment efficacy between the control group and AVD-001 groups using time to tumor endpoint (TTE). Log-rank two-tailed statistical analysis with a 95% confidence was used to determine the significance of tumor response comparisons between treatment groups and Logio cell kill analysis was used to indicate AVD-001 antitumor activity. Neither the TGD method of analysis nor the TTE method of analysis could be used for Group 2 (adriamycin control) since this group was discontinued early because of significant treatment-related body weight loss.

AVD-001 doses were generally well-tolerated in the mice, which gained weight progressively throughout the study. AVD-001 was administered to Group 3 animals without interruption, whereas AVD-001 administration in Groups 4 and 5 was temporarily suspended (Days 3 and 4 only) because of transient 10% body weight loss. Two animals in the escalating AVD-001 dose group (Group 5) were removed from the study (one on Day 34 and another on Day 38) because of drug toxicity, but the remaining Group 5 animals reached the 2000 mm³ tumor endpoint. All animals in Groups 1, 3, and 4 reached the 2000 mm³ tumor endpoint.

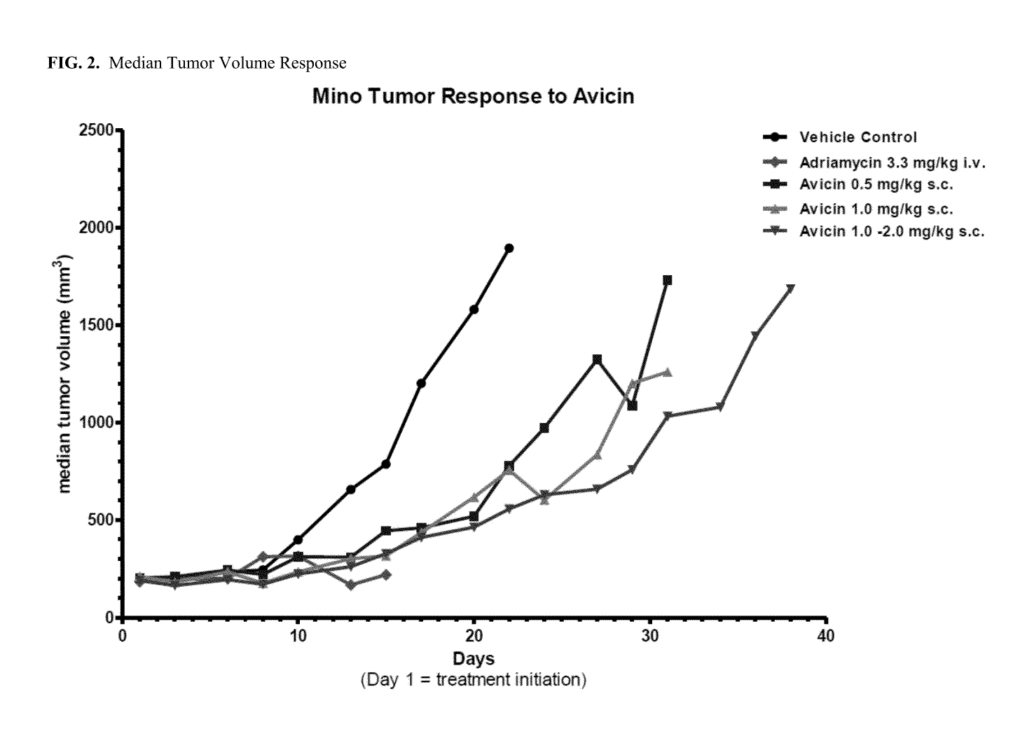

The escalating 1.0 to 2.0 mg/kg AVD-001 dose (Group 5) was most efficacious in inhibiting Mino tumor growth (FIG. 2). The corresponding 76.2% TGD was nearly double the 44.1% TGD for the 1.0 mg/kg dose (Group 4) and more than double the 34.9% TGD for the 0.5 mg/kg dose (Group 3) (Table 5).

TABLE 5
Tumor Response Summary

| | | AVD-001 | AVD-001 | AVD-001 |
|---|---|---|---|---|
| | Vehicle | 0.5 mg/kg | 1.0 mg/kg | 1.0 to 2.0 mg/kg |
| | (N = 9) | (N = 10) | (N = 10) | (N = 10) |
| Median TTE | 21.6 | 29.2 | 31.2 | 38.1 |
| % TGD | | 34.9 | 44.1 | 76.2 |
| CR | | — | — | — |
| PR | | 3 | 4 | 6 |
| Log₁₀ cell kill | | 0.57 | 0.45 | 0.89 |
| (750 mm³) | | | | |

CR = complete response;
PR = partial response;
TGD = tumor growth delay;
TTE = time to tumor endpoint.

Log-rank analysis of TGD showed significant AVD-001 therapeutic efficacy when compared to vehicle control, with Group 5 having the highest significance (p=0.0004) and Groups 3 (p=0.03) and 4 (p=0.02) having lower significance (Table 6).

TABLE 6
Efficacy Evaluation

| | | | |
|---|---|---|---|
| | Vehicle | Vehicle | Vehicle |
| | Control vs. | Control vs. | Control vs. |
| Treatments | AVD-001 | AVD-001 | AVD-001 |
| Compared | 0.5 mg/kg | 1.0 mg/kg | 1.0-2.0 mg/kg |

Log-rank (Mantel-Cox) Test

| | | | |
|---|---|---|---|
| p-value | 0.034 | 0.0236 | 0.0004 |

Median Survival (days)

| | | | |
|---|---|---|---|
| Group Control | 21.64 | 21.64 | 21.64 |
| AVD-001 Group | 29.2 | 31.18 | 38.14 |

Log-rank analysis of TGD also showed a significant dose response (p=0.004) between the escalating 1.0 to 2.0 mg/kg AVD-001 dose (Group 5) and the 0.5 mg/kg dose (Group 3). Log-rank analyses comparing AVD-001 treatment groups (i.e., Group 3 vs Group 4 and Group 4 vs. Group 5) did not show significant dose responses (p=0.3394 and p=0.3097, respectively).

In conclusion, monotherapy with AVD-001 resulted in significant TGD in Mino MCL xenograft bearing NODscid mice. There was high statistical significance when the escalating 1.0 to 2.0 mg/kg Avicin treatment response was compared to the vehicle control response. A Logio cell kill value of 0.89 in the escalating 1.0 to 2.0 mg/kg AVD-001 treatment group also indicated antitumor efficacy.

Efficacy on Mino Tumor Growth

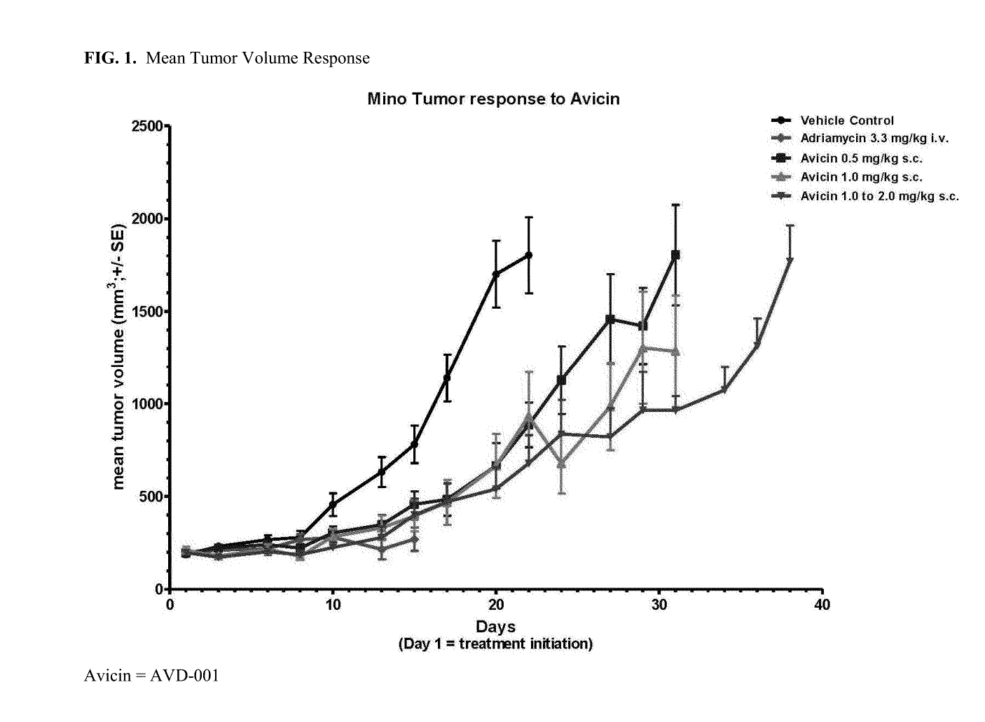

Tumors grew progressively in all treated and control animals through the 2000 mm³ tumor endpoint as shown by the mean tumor volume growth data in FIG. 1 and the median tumor volume growth data in FIG. 2. There were PR responses observed in treatment groups 3, 4, and 5. There were no CR or TFS responses observed for any treatment (Table 5).

Adriamycin 3.3 mg/kg: The tumor growth delay (TGD) method and time to tumor endpoint (TTE) could not be determined for the Adriamycin group (Group 2) as this group was discontinued early due to significant body weight loss as a result of treatment.

Avicin 0.5 mg/kg: This dose resulted in statistically significant tumor growth delay compared to vehicle control. Log-rank analysis showed that there was significant tumor response compared to vehicle control (p=0.03; Table 7). The median TTE was 29.2 days compared to 21.6 for vehicle control resulting in a 34.9% TGD and 3 PR responses were observed (Table 5). A Logio cell kill value of 0.57 for the 0.5 mg/kg Avicin treatment cohort was below the 0.7 threshold for indicating antitumor efficacy.

Avicin 1.0 mg/kg: This dose resulted in statistically significant tumor growth delay compared to vehicle control. Log-rank analysis showed that there was a significant tumor response compared to vehicle control (p=0.02; Table 7). The median TTE was 31.2 days compared to 21.6 days for vehicle control giving a 44.1% TGD and 4 PR responses were observed (Table 5). A Logio cell kill value of 0.45 for the 1.0 mg/kg Avicin treatment cohort was below the 0.7 threshold for indicating antitumor efficacy.

Avicin 1.0 to 2.0 mg/kg: This dose resulted in statistically significant tumor growth delay compared to vehicle control. Log-rank analysis showed that there was a significant tumor response compared to vehicle control (p=0.0004; Table 7). The median TTE was 38.1 days compared to 21.6 days for vehicle control giving a 76.2% TGD and 6 PR responses were observed (Table 5). A Logio cell kill value of 0.89 for the 1.0-2.0 mg/kg Avicin treatment cohort exceeded the 0.7 threshold for indicating antitumor efficacy.

Log-rank analysis also showed a significant dose response (p=0.004) between the escalating 1.0-2.0 mg/kg Avicin dose (Group 5) and the 0.5 mg/kg dose (Group 3). Log-rank analyses comparing Avicin treatment for Group 3 vs. Group 4 and Group 4 vs. Group 5 did not show significant dose responses (p=0.3394 and p=0.3097, respectively; Table 7).

Side Effects

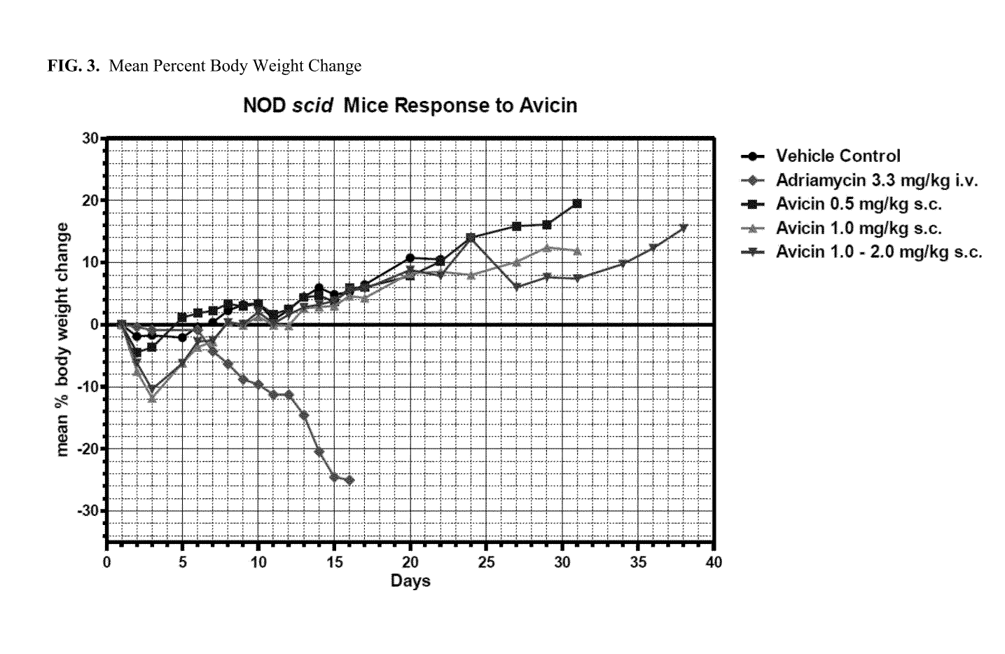

All three Avicin dosing protocols were generally well tolerated by the mice as indicated by progressive body weight gain throughout the study (FIG. 3 and Table 5). However, there was irritation and/or bleeding at the injection site for several Group 3, 4, and 5 mice. There were two treatment related deaths (TR) in the escalating Avicin dose Group 5. There were no non-treatment related deaths (NTR) during the study. The study animals were euthanized according to study protocol (EP) as each animal reached the 2000 mm³ study protocol tumor endpoint. All Group 5 animals with the exception of the two TR animals #2089 and #2062 reached the 2000 mm³ tumor endpoint. All animals in Groups 1, 3, and 4 reached the 2000 mm³ tumor endpoint (FIG. 1, FIG. 2).

Overall, mice progressively gained weight during the treatment course; however, the 0.5 mg/kg dose cohort had a nadir mean % body weight change of −4.43 on day 2 (FIG. 3). Avicin was administered without interruption in this group. The 1.0 mg/kg dose cohort had a nadir mean % body weight change of −11.82 on day 3. The escalating dose 1.0-2.0 cohorts had a nadir average % body weight change of −10.42 on day 3 and the vehicle control cohort had −2.09% nadir mean % body weight change. Drug administration in Groups 4 and 5 was temporarily suspended (day 3 and 4 only) because of the 10% transient body weight loss. These animals recovered and progressively gained weight following the transient weight loss. All mice received daily cage-side observation and all mice tolerated the treatments; clinical observations were bright, alert, responsive and hydrated.

The Adriamycin positive control animals experienced severe toxicity after the second drug dose and were removed from study by day 16.

### CONCLUSIONS

Monotherapy treatment with Avicin resulted in significant tumor growth delay in Mino mantle cell lymphoma xenograft bearing NOD scid mice. There was high statistical significance in comparisons between the escalating 1.0-2.0 mg/kg Avicin treatment response and the vehicle control.

Of the three Avicin test doses in this study, the escalating 1.0-2.0 mg/kg dose (Group 5) was most efficacious in inhibiting Mino tumor growth. The corresponding 76.2% TGD was nearly double the 44.1% TGD for the 1.0 mg/kg dose (Group 4) and more than double the 34.9% TGD for the 0.5 mg/kg dose (Group 3). Log-rank analysis showed significant Avicin therapeutic treatment efficacy compared to vehicle control, with Group 5 having the highest significance (p=0.0004) and Groups 3 (p=0.03) and 4 (p=0.02) having lower significance. In Group 5, 6 PR responses were observed and the Logio cell kill value was 0.89 which exceeded the 0.7 threshold for indicating antitumor efficacy. Logio cell kill values for Groups 3 and 4 were not significant although 3 and 4 PR responses were observed in these two Groups, respectively.

All three test doses were generally well tolerated as animals gained weight progressively throughout the study. However, drug administration in Groups 4 and 5 was temporarily suspended (day 3 and 4 only) because of 10% body weight loss. Mice in Group 3 experienced body weight loss of 4% which peaked at day 2, Avicin was administered to Group 3 animals without interruption. Two animals in the escalating Avicin dose group (Group 5) were removed from the study due to drug toxicity but the remaining animals in this group reached the tumor endpoint. All animals in groups 1, 3, and 4 reached the 2000 mm³ tumor endpoint. However, there was irritation and/or bleeding at the injection site for several Group 3, 4 and 5 mice. Additional data from these studies is shown in the figures and the tables below.

TABLE 7
AVD-001 Efficacy Evaluation

| | Group 1 vs. Group 3 | Group 1 vs. Group 4 | Group 1 vs. Group 5 |
|---|---|---|---|
| Groups Compared | Vehicle Control vs. | Vehicle Control vs. | Vehicle Control vs. |
| | Avicin 0.5 mg/kg s.c. | Avicin 1.0 mg/kg s.c. | Avicin 1.0-2.0 mg/kg s.c. |
| Log-rank (Mantel-Cox) Test | | | |
| Chi square | 4.494 | 5.121 | 12.38 |
| df | 1 | 1 | 1 |
| P value | 0.034 | 0.0236 | 0.0004 |
| P value summary | * | * | *** |
| Are the survival curves sig different? | Yes | Yes | Yes |
| Gehan-Breslow-Wilcoxon Test | | | |
| Chi square | 7.832 | 6.207 | 10.67 |
| df | 1 | 1 | 1 |
| P value | 0.0041 | 0.127 | 0.0011 |
| P value summary | ** | * | ** |
| Are the survival curves sig different? | Yes | Yes | Yes |
| Median survival | | | |
| Group Control | 21.64 | 21.64 | 21.64 |
| Group Treated | 29.2 | 31.18 | 38.14 |
| Ratio | 0.7412 | 0.6941 | 0.5675 |
| 95% CI of ratio | 0.3348 to 1.148 | 0.2971 to 1.091 | 0.817 to 0.9533 |
| Hazard Ratio | | | |
| Ratio | 3.159 | 3.492 | 10.36 |
| 95% CI of ratio | 1.091 to 9.147 | 1.182 to 10.31 | 2.816 to 38.10 |
| | Group 3 vs. Group 4 | Group 3 vs. Group 5 | Group 4 vs. Group 5 |
| Groups Compared | Avicin 0.5 mg/kg s.c. | Avicin 0.5 mg/kg s.c | Avicin 1.0 mg/kg s.c |
| | Avicin 1.0 mg/kg s.c. | Avicin 1.0-2.0 mg/kg s.c. | Avicin 1.0-2.0 mg/kg s.c. |
| Log-rank (Mantel-Cox) Test | | | |
| Chi square | 0.9125 | 8.005 | 1.032 |
| df | 1 | 1 | 1 |
| P value | 0.3394 | 0.0047 | 0.3097 |
| P value summary | ns | ** | ns |
| Are the survival curves sig different? | No | Yes | No |
| Gehan-Breslow-Wilcoxon Test | | | |
| Chi square | 0.1412 | 4.721 | 2.262 |
| df | 1 | 1 | 1 |
| P value | 0.707 | 0.0298 | 0.1326 |
| P value summary | ns | * | ns |
| Are the survival curves sig different? | No | Yes | No |
| Median survival | | | |
| Group Control | 29.2 | 29.2 | 31.18 |
| Group Treated | 31.18 | 38.14 | 38.14 |
| Ratio | 0.9365 | 0.7657 | 0.8177 |
| 95% CI of ratio | 0.5301 to 1.343 | 0.3710 to 1.160 | 0.4318 to 1.203 |
| Hazard Ratio | | | |
| Ratio | 1.588 | 5.491 | 1.674 |
| 95% CI of ratio | 0.6148 to 4.103 | 1.688 to 17.87 | 0.6195 to 4.524 |

ns = non significant
* = P ≦ 0.05
** = P ≦ 0.01
*** = P ≦ 0.001

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Baran-Marszak F, Boukhiar M, Harel S, Laguillier C, Roger C, Gressin R, et al. Constitutive and B-cell receptor-induced activation of STAT3 are important signaling pathways targeted by bortezomib in leukemic mantle cell lymphoma. Haematologica 2010;95(11):1865-1872.

Beltran E, Fresquet V, Martinez-Useros J, Richter-Larrea J A, Sagardoy A, Sesma I, et al. A cyclin-D 1 interaction with BAX underlies its oncogenic role and potential as a therapeutic target in mantle cell lymphoma. Proc Natl Acad Sci USA 2011;108(30):12461-12466.

Blackstone N W, Kelly M M, Haridas V, Gutterman J U. Mitochondria as integrators of information in an early-evolving animal: insights from a triterpenoid metabolite. Proc Biol Sci 2005;272(1562):527-531.

de Boer C J, van Krieken J H, Kluin-Nelemans H C, Kluin P M, Schuuring E. Cyclin D1 messenger RNA overexpression as a marker for mantle cell lymphoma. Oncogene 1995;10(9):1833-1840.

Dreyling M, Hiddemann W. Current treatment standards and emerging strategies in mantle cell lymphoma. Hematology Am Soc Hematol Educ Program 2009:542-551.

EMA Committee for Orphan Medicinal Products (COMP). Minutes of the 5-6 Feb. 2013 Meeting. 2013. Human Medicines Development and Evaluation. EMA/COMP/18213/2013.

Gaikwad A, Poblenz A, Haridas V, Zhang C, Duvic M, Gutterman J. Triterpenoid electrophiles (avicins) suppress heat shock protein-70 and x-linked inhibitor of apoptosis proteins in malignant cells by activation of ubiquitin machinery: implications for proapoptotic activity. Clin Cancer Res 2005;11(5):1953-1962.

Gutterman J U, Lai H T, Yang P, Haridas V, Gaikwad A, Marcus S. Effects of the tumor inhibitory triterpenoid avicin G on cell integrity, cytokinesis, and protein ubiquitination in fission yeast. Proc Natl Acad Sci U S A 2005;102(36):12771-12776.

Hall L. Efficacy of novel compounds in a mouse model of collagen-induced arthritis. In Vivo Services, The Jackson Laboratory West. Study number: IVS16783. 2009.

Hall L. Investigation of the interaction of Avicin D and LPS in mice. In Vivo Services, The Jackson Laboratory-West. Study number: IVS 18553. 2010.

Haridas V, Arntzen C J, Gutterman J U. Avicins, a family of triterpenoid saponins from Acacia victoriae

(Bentham), inhibit activation of nuclear factor-kappaB by inhibiting both its nuclear localization and ability to bind DNA. Proc Natl Acad Sci U S A 2001;98(20):11557-11562.

Haridas V, Hanausek M, Nishimura G, Soehnge H, Gaikwad A, Narog M, et al. Triterpenoid electrophiles (avicins) activate the innate stress response by redox regulation of a gene battery. J Clin Invest 2004;113(1):65-73.

Haridas V, Higuchi M, Jayatilake G S, Bailey D, Mujoo K, Blake M E, et al. Avicins: triterpenoid saponins from Acacia victoriae (Bentham) induce apoptosis by mitochondrial perturbation. Proc Natl Acad Sci U S A 2001;98(10):5821-5826.

Haridas V, Kim S O, Nishimura G, Hausladen A, Stamler J S, Gutterman J U. Avicinylation (thioesterification): a protein modification that can regulate the response to oxidative and nitrosative stress. Proc Natl Acad Sci U S A 2005;102(29):10088-10093.

Haridas V, Li X, Mizumachi T, Higuchi M, Lemeshko V V, Colombini M, et al. Avicins, a novel plant-derived metabolite lowers energy metabolism in tumor cells by targeting the outer mitochondrial membrane. Mitochondrion 2007;7(3):234-240.

Haridas V, Nishimura G, Xu Z X, Connolly F, Hanausek M, Walaszek Z, et al. Avicin D: a protein reactive plant isoprenoid dephosphorylates Stat 3 by regulating both kinase and phosphatase activities. PLoS One 2009;4(5):e5578.

Harris N L, Jaffe E S, Stein H, Banks P M, Chan J K, Cleary M L, et al. A revised European-American classification of lymphoid neoplasms: a proposal from the International Lymphoma Study Group. Blood 1994;84(5):1361-1392.

Hernandez L, Bea S, Pinyol M, Ott G, Katzenberger T, Rosenwald A, et al. CDK4 and MDM2 gene alterations mainly occur in highly proliferative and aggressive mantle cell lymphomas with wild-type INK4a/ARF locus. Cancer Res 2005;65(6):2199-2206.

Hernandez L, Fest T, Cazorla M, Teruya-Feldstein J, Bosch F, Peinado M A, et al. p53 gene mutations and protein overexpression are associated with aggressive variants of mantle cell lymphomas. Blood 1996;87(8):3351-3359.

Hoster E, Dreyling M, Klapper W, Gisselbrecht C, van Hoof A, Kluin-Nelemans H C, et al. A new prognostic index (MPI) for patients with advanced-stage mantle cell lymphoma. Blood 2008;111(2):558-565.

Jaffe E S, Harris H, Stein J W. World Health Organization Classification of Tumors: Pathology and Genetics of Tumors of Haematopoietic and Lymphoid Tissues 2001;

Jares P, Colomer D, Campo E. Molecular pathogenesis of mantle cell lymphoma. J Clin Invest 2012;122(10):3416-3423.

Jayatilake G S, Freeberg D R, Liu Z, Richheimer S L, Blake Nieto M E, Bailey D T, et al. Isolation and structures of avicins D and G: in vitro tumor-inhibitory saponins derived from Acacia victoriae. J Nat Prod 2003;66(6):779-783.

Lai R, Rassidakis G Z, Medeiros U, Leventaki V, Keating M, McDonnell T J. Expression of STAT3 and its phosphorylated forms in mantle cell lymphoma cell lines and tumors. J Pathol 2003;199(1):84-89.

Lemeshko V V, Haridas V, Quijano Perez J C, Gutterman J U. Avicins, natural anticancer saponins, permeabilize mitochondrial membranes. Arch Biochem Biophys 2006;454(2):114-122.

Leux C, Maynadie M, Troussard X, Cabrera Q, Herry A, Le Guyader-Peyrou S, et al. Mantle cell lymphoma epidemiology: a population-based study in France. Ann Hematol 2014; 93(8):1327-33.

Mitsiades N, McMullan C J, Poulaki V, Negri J, Geer D C, Haridas V, et al. Avicins: a novel class of anti-myeloma agents. ASH Annual Meeting Abstracts. Blood 2004:Abst No 3405.

Mitterlechner T, Fiegl M, Mühlböck, Oberaigner W, Dirnhofer, Tzankov A. Epidemiology of non-Hodgkin lymphomas in Tyrol/Austria from 1991 to 2000. J Clin Pathol 2006;59:48-55.

Mujoo K, Haridas V, Hoffmann J J, Wachter G A, Hutter L K, Lu Y, et al. Triterpenoid saponins from Acacia victoriae (Bentham) decrease tumor cell proliferation and induce apoptosis. Cancer Res 2001;61(14):5486-5490.

Pham L V, Tamayo A T, Yoshimura L C, Lo P, Ford R J Inhibition of constitutive NF-kappa B activation in mantle cell lymphoma B cells leads to induction of cell cycle arrest and apoptosis. J Immunol 2003;171(1):88-95.

Pinyol M, Bea S, Pla L, Ribrag V, Bosq J, Rosenwald A, et al. Inactivation of RB1 in mantle-cell lymphoma detected by nonsense-mediated mRNA decay pathway inhibition and microarray analysis. Blood 2007;109(12):5422-5429.

Roue G, Perez-Galan P, Lopez-Guerra M, Villamor N, Campo E, Colomer D. Selective inhibition of IkappaB kinase sensitizes mantle cell lymphoma B cells to TRAIL by decreasing cellular FLIP level. J Immunol 2007;178(3):1923-1930.

Sant M, Allemani C, Tereanu C, De Angelis R, Capocaccia R, Visser O, et al. Incidence of hematologic malignancies in Europe by morphologic subtype: results of the HAEMACARE project. Blood 2010;116(19):3724-3734.

Smedby K E, Hjalgrim H. Epidemiology and etiology of mantle cell lymphoma and other non-Hodgkin lymphoma subtypes. Semin Cancer Biol 2011;21(5):293-298.

Xu Z X, Liang J, Haridas V, Gaikwad A, Connolly F P, Mills G B, et al. A plant triterpenoid, avicin D, induces autophagy by activation of AMP-activated protein kinase. Cell Death Differ 2007;14(11):1948-1957.

Zhang C, Li B, Gaikwad A S, Haridas V, Xu Z, Gutterman J U, et al. Avicin D selectively induces apoptosis and downregulates p-STAT-3, bcl-2, and survivin in cutaneous T-cell lymphoma cells. J Invest Dermatol 2008;128(11):2728-2735.

1. A method of treating mantle cell lymphoma (MCL) in a subject in need of said treatment, comprising administering a pharmacologically effective amount of avicin D to the subject, wherein the subject has mantle cell lymphoma (MCL).

2. The method of claim 1, wherein the subject is a mammal.

3. The method of claim 2, wherein the mammal is a human.

4. The method of claim 1, wherein said amount is from about 0.0125 mg/kg to about 1 mg/kg.

5. The method of claim 4, wherein said amount is from about 0.025 mg/kg to about 0.75 mg/kg.

6. The method of claim 4, wherein said amount is from about 0.0125 mg/kg to about 0.5 mg/kg.

7. The method of claim 6, wherein said amount is from about 0.0125 mg/kg to about 0.1 mg/kg.

8. The method of claim 1, wherein the avicin D is comprised in a pharmaceutically acceptable excipient or diluent.

9. The method of claim 8, wherein the pharmaceutically acceptable excipient or diluent is formulated for injection or oral administration.

10. The method of claim 9, wherein said injection is intravenous (i.v.), subcutaneous (s.q.), intracutaneous (i.c.), intramuscular (i.m.), or intraperitoneal (i.p.).

11. The method of claim 1, wherein said avicin D is administered prior to, after, or in combination with another anti-cancer agent, an anti-cancer therapy, or a surgery.

12. The method of claim 11, wherein the anti-cancer agent is a chemotherapeutic.

13. The method of claim 12, wherein the anti-cancer agent is cyclophosphamide, hydrozydaunorubicin, vincristine, prednisone, rituximab, cytarabine, dexamethasone, cytarabine, cisplatin, or bendamustine.

14. The method of claim 11, wherein said avicin D is administered prior to, after, or in combination with an anti-cancer therapy, wherein the anti-cancer therapy is CHOP, R-CHOP, R-bendamustine, DHAP, R-DHAP, or R-Hyper-CVAD/MA.

15. The method of claim 1, wherein said MCL in said subject is resistant to a chemotherapy.

16. The method of claim 15, wherein the chemotherapy is cyclophosphamide, hydrozydaunorubicin, vincristine, prednisone, rituximab, cytarabine, dexamethasone, cytarabine, cisplatin, or bendamustine.

17. The method of claim 1, wherein said MCL in said subject is resistant to an anti-cancer therapy.

18. The method of claim 17, wherein the anti-cancer therapy is CHOP, R-CHOP, R-bendamustine, DHAP, R-DHAP, or R-Hyper-CVAD/MA.

19. The method of claim 1, wherein the method is further defined as a method of overcoming chemotherapeutic resistance of said MCL to an anti-cancer treatment.

20. The method of claim 1, wherein growth rates of tumors of said MCL in the subject decrease.

21. The method of claim 1, wherein the total tumor volume resulting from said MCL in the subject decrease.

22. The method of claim 1, wherein said MCL is a classic MCL or a nodular MCL.

23. The method of claim 1, wherein said MCL is a diffuse MCL or a blastoid MCL.

24. The method of claim 1, wherein avicin D is administered to the subject in an amount of from about 0.0125 to about 0.1 mg/kg per day.

25. The method of claim 24, wherein the avicin D is administered intravenously or subcutaneously.

26-30. (canceled)
